(19) United States
(12) Patent Application Publication
(10) Pub. No.: US 2022/0175533 A1
(43) Pub. Date: Jun. 9, 2022

(54) NESTED HARD TISSUE REPLACEMENT IMPLANTS

(71) Applicant: Zimmer Biomet CMF and Thoracic, LLC, Jacksonville, FL (US)

(72) Inventors: Max Holland Billard, Jacksonville, FL (US); Zachary Frank, Jacksonville, FL (US); Laura Zborowski, Jacksonville, FL (US); Adam Webster Hausman, Jacksonville, FL (US)

(21) Appl. No.: 17/534,856

(22) Filed: Nov. 24, 2021

Related U.S. Application Data
(60) Provisional application No. 63/120,869, filed on Dec. 3, 2020.

(51) Int. Cl. A61F 2/28; A61B 17/80

(52) U.S. Cl. CPC A61F 2/2875; A61F 2002/2835; A61B 17/8061

(57) ABSTRACT

A method of replacing a portion of a cranium with a hard tissue implant can include resecting a first portion of the cranium to define a first margin region. The first portion of the cranium can be analyzed to determine if the first margin region is free of cancerous cells. A second portion of the cranium can be resected to define a second margin region that is larger than the first margin region when the first margin region is not free of cancerous cells. An outer implant can be secured to the cranium at the second margin region. An inner implant can be secured to the outer implant.

### CLAIM OF PRIORITY

This patent application claims the benefit of priority, under 35 U.S.C. Section 119(e), to Billard, U.S. Patent Application Ser. No. 63/120,869, entitled “NESTED HARD TISSUE REPLACEMENT IMPLANTS,” filed on Dec. 3, 2020 (Attorney Docket No. 4394.Q73PRV), which is hereby incorporated by reference herein in its entirety.

### BACKGROUND

Hard tissue replacement implants for areas such as bone are often required to replace sections of bone damaged by tumors or infection. For example, hard tissue replacements can be used to replace sections of cranium or skull when tumorous portions of the cranium are removed. The sections can be replaced by implants such as plates that can be secured to the remaining healthy skull to cover the void left by removing compromised bone.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a side perspective view of a skull.

FIG. 1B illustrates a side isometric view of a skull.

FIG. 2A illustrates a top view of a hard tissue replacement implant.

FIG. 2B illustrates a top view of a hard tissue replacement implant.

FIG. 2C illustrates a cross-sectional view of an implant and a cranium.

FIG. 3A illustrates a top view of a hard tissue replacement implant.

FIG. 3B illustrates a top view of a hard tissue replacement implant.

FIG. 4 illustrates a top view of a hard tissue replacement implant.

FIG. 5 illustrates a top view of a hard tissue replacement implant.

FIG. 6 illustrates a flow schematic for installing a hard tissue implant.

### DETAILED DESCRIPTION

Hard tissue replacement implants for areas such as bone are often required to replace sections of bone damaged by tumors or infections. During one type of procedure, a damage section is removed to an estimated first margin and one or more samples are taken to determine if the first margin is free of cancerous cells. If it is, an implant can be secured to the cranium (or bone) to replace the resected bone. If the margin is not free of cancerous cells, the surgeon typically goes back to resect more bone to a second margin and samples are tested again. The process can be repeated to ensure the margins are all free of cancerous cells. Because this process can be time consuming and can be costly due to acquiring a set of implants of various sizes, surgeons often make a conservative operative plan to ensure that the margins will be free of cancerous cells after the first resection and one implant size can be ordered. However, while this plan can save time and cost, such a conservative approach can result in more healthy bone being taken than is required.

The inventors have recognized that this procedure can be modified to potentially save more healthy bone by using a nesting hard tissue replacement implant assembly. The first (inner) implant can be selected for replacement of a resected bone at a first margin according to a pre-operative plan where the first implant is sized to replace the first resected portion at the first margin, where the first margin is likely to be cancer-free but where the first margin is less conservative than one of the conservative approach of the strategy discussed above. Following resection, edge samples can be analyzed to ensure that the edges are free of cancerous cells.

When analysis confirms that the margins are safe and free of cancerous cells, the inner implant can be installed to replace the resected portion. However, when the analysis determines that, the margins are not free of cancerous cells, the surgeon can resect the bone a second time to create a second margin that is larger than the first margin, such as according to a pre-operative plan where the second margin is selected to be more conservative and very likely to be free of cancerous cells. The outer implant can then be secured to the cranium (bone) and the inner implant can be nested within the outer implant and secured to the outer implant before completing a normal closing procedure. Such a process can help to save as much healthy bone as the surgeon thinks is possible while helping to minimize iteration of resection and testing and while using a single implant system to help reduce costs.

Though the invention is discussed with respect to cancerous bone replacement. The same methods can be applied for infected bone requiring replacement.

The above discussion is intended to provide an overview of subject matter of the present patent application. It is not intended to provide an exclusive or exhaustive explanation of the invention. The description below is included to provide further information about the present patent application.

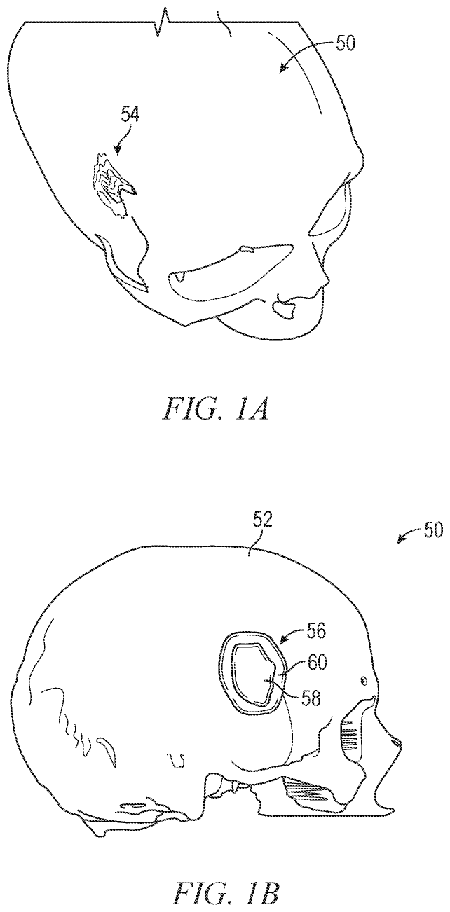

FIG. 1A illustrates a side perspective view of a skull 50 including a cranium bone portion 52 and a tumorous portion 54 of the cranium 52. FIG. 1B illustrates a side isometric view of the skull 50 showing margin plan 56 for the procedure. The margin plan 56 can include an inner margin portion 58 that is likely to be free of cancerous cells and is sized to receive an inner implant. The margin plan 56 can include an outer margin portion 60 that is larger than the inner margin 58 and is sized to receive an outer implant. Which implant is used can be based on edge sample analysis, as discussed in further detail below.

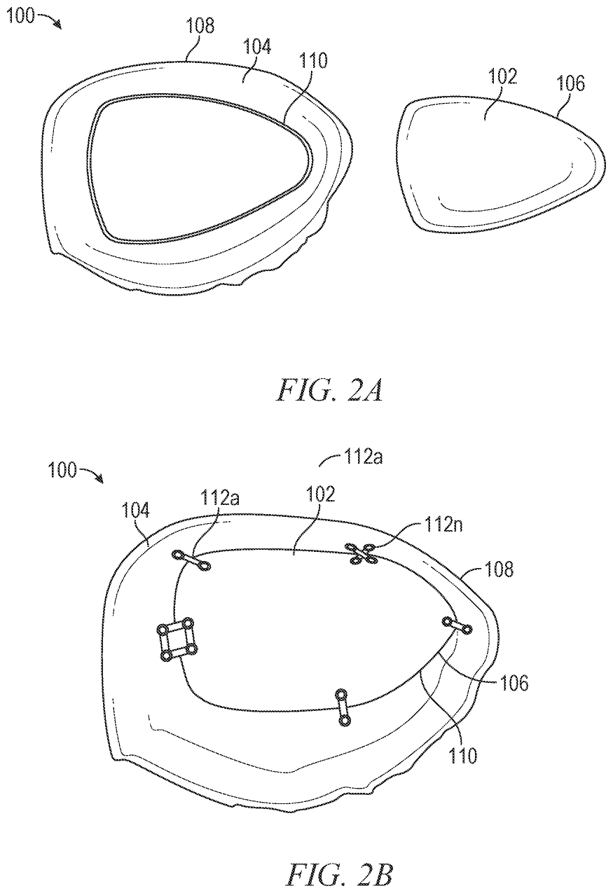

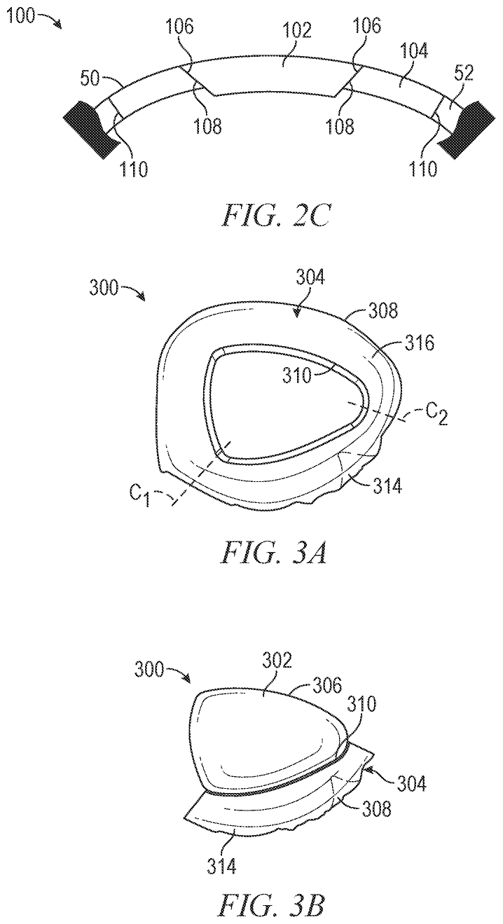

FIG. 2A illustrates a top view of a hard tissue replacement implant assembly 100 (referred to as implant assembly 100). FIG. 2B illustrates a top view of the hard tissue replacement implant assembly 100. FIG. 2C illustrates a cross-sectional view of the implant assembly 100 and a cranium 52. FIGS. 2A-2C are discussed together below.

The implant assembly 100 can include an inner (or first) implant 102 and an outer (or second) implant 104. The inner implant can define an outer edge 106. The outer implant 104 can define an outer edge 108 and an inner edge 110. The inner implant 102 and the outer implant 104 can be made of biocompatible materials such as one or more of stainless steels, cobalt-chromium, titanium variations, polyether ketone ketone (PEKK), polyether ether ketone (PEEK), or the like.

The implant assembly 100 can be designed in a pre-operative planning procedure where a surgeon or physician selects a first margin region (such as the region 58 of FIG. 1B), that is likely to be free of cancerous cells. The physician can then select an outer margin region, such as the margin portion 60 of FIG. 1B, that is larger than the inner margin 58 and very likely to be free of cancerous cells. The inner implant 102 can be sized to replace hard tissue of the inner region 58 and the outer implant 104 can be sized such that the inner implant 102 can nest in the outer implant 104 and such that the inner implant 102 and outer implant 104 can be sized to, together, replace all of the hard tissue removed to create the outer margin 60. Such an implant assembly can help to preserve as much healthy bone as the surgeon thinks is possible while helping to minimize iteration of resection and testing and while using a single implant system to help reduce costs.

As shown in FIGS. 2A and 2C, the inner edge 110 of the outer implant 104 can be beveled. And, as sown in FIG. 2C, the outer edge 106 of the inner implant can be beveled (or chamfered) complimentary to the bevel of the inner edge 110 of the outer implant 104. The bevels of the outer edge 106 and the inner edge 110 can help to allow the inner implant 102 to nest within the outer implant 104, where outer surfaces of the inner implant 102 and the outer implant 104 are aligned or even where the inner implant 102 and the outer implant 104 meet, as shown in FIGS. 2B and 2C. The bevels of the outer edge 106 and the inner edge 110 can also be configured (angled down and radially inwardly) to limit movement of the inner implant 102 through the inner implant 104 when the implants are in the nested orientation of FIG. 2B, which can help limit relatively movement of the implants and can help to enable simpler fastening of the implants.

As shown in FIG. 2B, the implants 102 and 104 can be secured together using fasteners 112a-112n, where the fasteners 112 can be secured to both the inner implant 102 and the outer implant 104. The fasteners 112 can also be used to secure the outer implant 104 to bone (such as the cranium 52). The fasteners 112 can be bone fasteners of any type (e.g., screws, plates, or pins).

FIG. 3A illustrates a top view of a hard tissue replacement implant assembly 300. FIG. 3B illustrates a top view of the hard tissue replacement implant assembly 300 (implant assembly 300). FIGS. 3A and 3B are discussed together below.

The implant assembly 300 can include an inner (or first) implant 302 and an outer (or second) implant 304. The inner implant can define an outer edge 306. The outer implant 304 can define an outer edge 308 and an inner edge 310. The outer implant 304 can also include a trimmed portion 314 and a sacrificial portion 316. Also shown in FIG. 3A are cut lines C1 and C2.

The implant assembly 300 of FIGS. 3A and 3B can be similar to the implant assembly 100 discussed above with respect to FIGS. 2A-2C; FIGS. 3A and 3B show another way that such implant assemblies can be used. For example, after the first margin resection is performed it may be determined that only a portion of the second margin must be removed. In such a case, the outer implant 304 can be trimmed to match the smaller resected second margin, such as by trimming the outer implant at cut lines C1 and C2. Cut lines C1 and C2 can be straight, which can help to simplify the implant trimming process and the bone resection process.

Then, the trimmed portion 314 of the outer implant 304 can be secured to bone to fill the void created by the second margin portion. The inner implant 302 can then be secured to the trimmed portion 314 and the cranium to fill the remainder of the void created by creation of the first margin portion. The sacrificial portion 316 can be discarded. Such a process can allow for a single implant assembly to be used to help fill voids of a variety of margin shapes and locations helping to conserve as much healthy bone as possible.

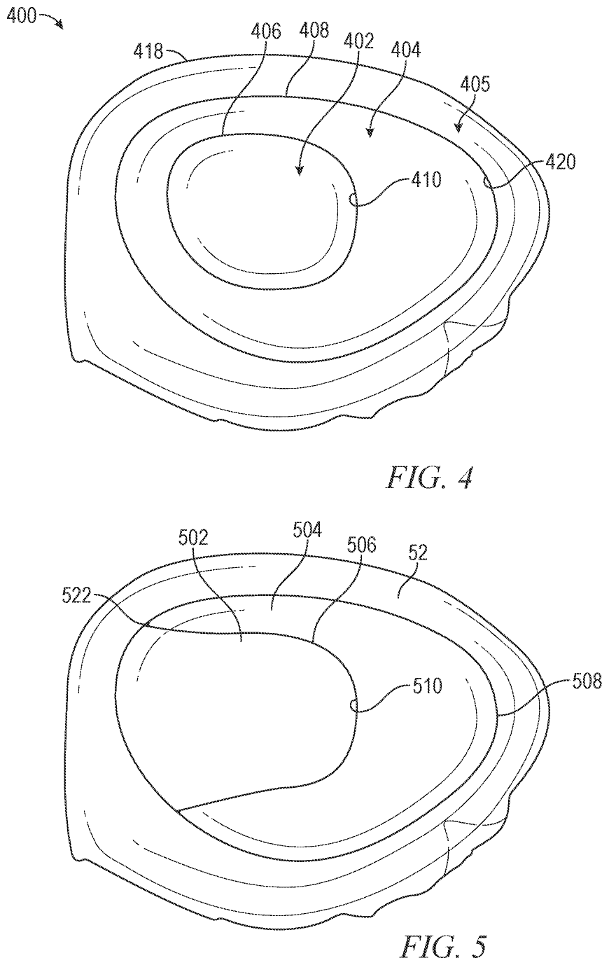

FIG. 4 illustrates a top view of a hard tissue replacement implant assembly 400. The implant 400 can include an inner (or first) implant 402 a middle (or second) implant 404, and an outer (or third) implant 405. The inner implant 402 can define an outer edge 406. The middle implant 404 can define an outer edge 408 and an inner edge 410. The outer implant 405 can define an outer edge 418 and an inner edge 420.

The implant assembly 400 can be similar to the implant assemblies 100 and 300 discussed above; the implant assembly 400 can include a third implant (the outer implant 405. Such an implant assembly can allow for a third margin region to be planned for in a case where a surgeon is trying to conserve as much bone as possible or where there is a lot of uncertainty as to where safe margins will be (for example due to a rapidly expanding cancer or infection). For example, the inner implant 402 can be used when the first margin region is free of cancerous cells, the inner implant 402 and at least a portion of the middle implant 404 can be used when the first margin region is not free of cancerous cells, and the inner implant 402 and at least a portion of the middle implant 404 and the outer implant 405 can be used when the second margin region is not free of cancerous cells.

The inner implant 402 can be configured to nest within the middle implant 404 and the middle implant 404 can be configured to nest within the outer implant 405 such that the inner implant 402 and the middle implant 404 nest within the outer implant 405. The edges (406, 408, 410, and 420) of the implants can all be beveled or chamfered (similar to the implant assembly 100) to allow for nesting. Such a nesting implant assembly can allow the implants to act as a single implant when secured to each other. FIG. 4 also shows that the implants 402 and 404 can have irregular and different shapes.

Also, in some examples, the implant assembly 400 can be manufactured to a standard size. For example, a three-piece assembly 400 can be manufactured to match contours of an average person's cranium for a portion (such as a lateral portion) using, for example, a dataset of dimensions for a large number of samples. The samples can be averaged and the inner implant 402, middle implant 404, and the outer implant 405 can each be manufactured based on the data. Such an implant assembly can be kept in stock for emergency procedures or can be used where cost reduction is more important than bone conservation. In some examples, average tumor or infection size and growth rate can be used to create the inner implant 402, middle implant 404, and the outer implant 405.

FIG. 5 illustrates a top view of a hard tissue replacement implant assembly 500. The implant assembly 500 can include an inner (or first) implant 502 and an outer (or second) implant 304. The inner implant can define an outer edge 506. The outer implant 504 can define an outer edge 508 and an inner edge 510.

The implant assembly 500 of FIG. 5 can be similar to the implant assemblies 100 and 300 discussed above; the outer implant 504 can form a U-shape. More specifically, the inner edge 510 of the outer implant can meet the outer edge 508 such that the outer implant 504 can include an opening 522 on one side such that the outer implant 504 forms a C-shape or a U-shape. The exact shape can vary such that the outer implant 504 is not a precise C-shape or U-shape but is not a complete shape such that it is open to one side allowing the outer edge 506 of the inner implant 502 to form a portion of an outer periphery of the implant assembly 500. Such an assembly can be useful where margins are less certain on one or more portions of the cranium 52.

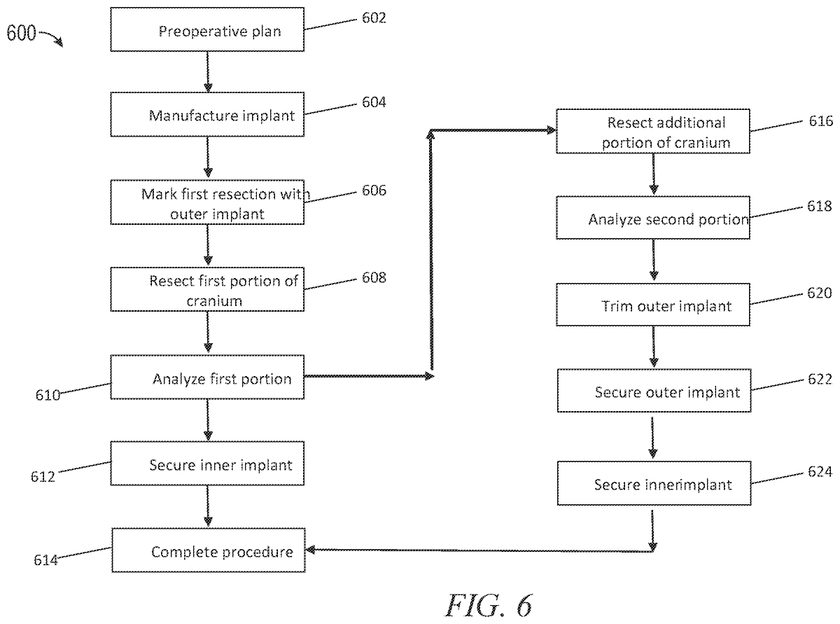

FIG. 6 illustrates a schematic view of the method 600, in accordance with at least one example of this disclosure. The method 600 can be a method of securing an implant assembly to bone. More specific examples of the method 600 are discussed below. The steps or operations of the method 600 are illustrated in a particular order for convenience and clarity; many of the discussed operations can be performed in a different sequence or in parallel without materially impacting other operations. The method 600 as discussed includes operations performed by multiple different actors, devices, and/or systems. It is understood that subsets of the operations discussed in the method 600 can be attributable to a single actor, device, or system could be considered a separate standalone process or method.

At step 602 a preoperative plan can be created, such as by a physician or surgeon where the inner implant (such as the implant 102) can be selected for replacement of a resection at a first margin where the first implant size is selected at a first margin that is likely to be cancer-free but with margins less conservative than in a second margin region. The outer implant (such as the implant 104) can be sized to replace bone of the second margin region. Similarly, a third, further, etc., implant can be sized for margins during the preoperative planning step.

At step 604 the implant can be manufactured based on the preoperative plan and the patient's anatomy such as by using a three-dimensional (3D) printing procedure (e.g., selective laser sintering). Such a manufacturing procedure can help reduce the likelihood of warping or mushrooming that can occur in the fabrication of a single large implant. The use of 3D printing also helps to reduce material waste over other machining alternatives.

The surgical procedure can begin at step 606 after the patient is prepped and an opening is created. At step 606 an inner edge of the outer implant (such as the edge 110) can be used to mark the first resection location on the cranium. Marking the resection in this manner can be beneficial because deformed portions (such as a protruding tumor) of the cranium can extend through the opening of the outer implant (e.g., opening of the outer implant 104), which can help to allow the resection lines for the first margin to be marked more accurately than by use of a flat template. At step 608 the first portion of the cranium can be resected to the first margin portion and at step 610 edge samples can be analyzed to determine whether they are free of cancerous cells.

When the samples are determined to be free of cancerous cells (and the first margin portion is therefore good or clear), step 612 can be performed where the inner implant (e.g., the inner implant 102) can be secured to the cranium and the procedure can be completed. When the edge samples are not free of cancerous cells, a second portion of the cranium can be resected at step 616. A portion of the second margin area can be resected or all of the area can be resected based on the results of the analysis from step 610. Then, samples (such as edge samples) from the second resection can be analyzed to determine if they are free of cancerous cells. If so, the outer implant (e.g., 304) can be trimmed at the step 620 based on the results and the trimmed portion (e.g., trimmed portion 314) can be secured to the cranium at step 622. Optionally, such as when multiple edge samples include tumorous cells, the entire second margin region can be removed and trimming of the outer implant can be skipped and the entire outer implant (e.g., 304) can be secured to the cranium at step 622. When the analysis of step 618 determines that the additional portion of the resection is not free of cancerous cells, the steps 616 and 618 can be repeated until margins free of cancerous cells is achieved.

Once the outer implant(s) are secured to the cranium, the inner implant(s) (e.g., 102, 302, or 402) can be nested within the outer implant and secured thereto. When a middle implant or implants is used, the middle implant(s) can be secured to the outer implant prior to securing the inner implant. Any of the implants can be secured using a plurality of fasteners. Then, at step 614, following either of steps 612 or 624, the procedure can be completed (including closing, etc.).

Such a procedure can help to save as much healthy bone as the surgeon thinks is possible while helping to minimize iteration of resection and testing and while using a single implant system to help reduce costs. Also, by using a smaller implant as the inner implant, surgeons are provided with a smaller access area for reentry, if required. Though the devices and procedures are discussed primarily with respect to replacement of cancerous cranium segments, they can be applied to replacement of infected bone or for replacement of fractured or deformed bone in any location.

### NOTES AND EXAMPLES

The following, non-limiting examples, detail certain aspects of the present subject matter to solve the challenges and provide the benefits discussed herein, among others.

Example 1 is a method of replacing a portion of a cranium with a hard tissue implant, the method comprising: resecting a first portion of the cranium to define a first margin region; analyzing the first portion of the cranium to determine if the first margin region is free of cancerous cells; resecting a second portion of the cranium to define a second margin region that is larger than the first margin region when the first margin region is not free of cancerous cells; securing an outer implant to the cranium at the second margin region; and securing an inner implant to the outer implant.

In Example 2, the subject matter of Example 1 optionally includes wherein the outer implant is secured to the inner implant using a plurality of fasteners.

In Example 3, the subject matter of any one or more of Examples 1-2 optionally include nesting the inner implant in the outer implant.

In Example 4, the subject matter of Example 3 optionally includes wherein an inner edge of the outer implant is beveled and an outer edge of the inner implant is beveled complimentary to the beveled inner edge of the outer implant to allow the inner implant to nest in the outer implant.

In Example 5, the subject matter of any one or more of Examples 1-4 optionally include trimming the outer implant based on the analysis; and securing the trimmed outer implant to the cranium.

In Example 6, the subject matter of Example 5 optionally includes securing the inner implant to the trimmed outer implant and to the cranium.

In Example 7, the subject matter of any one or more of Examples 5-6 optionally include wherein the outer implant is U-shaped.

In Example 8, the subject matter of any one or more of Examples 1-7 optionally include marking, using the outer implant as a guide, the cranium for resecting the first portion of the cranium to the first margin region.

In Example 9, the subject matter of any one or more of Examples 1-8 optionally include wherein the inner implant and the outer implant are manufactured through selective laser sintering.

Example 10 is a method of replacing a portion of a cranium with a hard tissue implant, the method comprising: marking the cranium for resecting a first portion of the cranium to define a first margin region using an inner edge of an outer implant as a guide; resecting the first portion of the cranium to the first margin region; analyzing the first portion of the cranium to determine if the first margin region is free of cancerous cells; and securing an inner implant to the cranium when the first margin region is free of cancerous cells.

In Example 11, the subject matter of Example 10 optionally includes resecting a second portion of the cranium to define a second margin region that is larger than the first margin region when the first margin region is not free of cancerous cells; and securing an outer implant to the cranium at the second margin region.

In Example 12, the subject matter of Example 11 optionally includes securing an inner implant to the outer implant when the first margin region is not free of cancerous cells.

In Example 13, the subject matter of Example 12 optionally includes wherein the outer implant is secured to the inner implant using a plurality of fasteners,

In Example 14, the subject matter of any one or more of Examples 12-13 optionally include nesting the inner implant in the outer implant.

In Example 15, the subject matter of Example 14 optionally includes wherein an inner edge of the outer implant is beveled and an outer edge of the inner implant is beveled complimentary to the beveled inner edge of the outer implant to allow the inner implant to nest in the outer implant.

In Example 16, the subject matter of Example 15 optionally includes wherein the outer implant is U-shaped.

Example 17 is a hard tissue implant assembly comprising: an outer portion securable to a cranium of a patient, the outer portion including an inner edge that is beveled; and an inner portion including a beveled outer edge engageable with the inner edge of the outer portion to nest the inner portion within the outer portion, the inner portion securable to the outer portion.

In Example 18, the subject matter of Example 17 optionally includes a plurality of fasteners securable to the inner portion and the outer portion to secure the inner portion to the outer portion and to secure the outer portion to the cranium.

In Example 19, the subject matter of any one or more of Examples 17-18 optionally include wherein the outer implant is trimmable to reduce an overall shape and size of the outer implant, the portion of the outer implant securable to the cranium, and the inner implant securable to the portion of the outer implant and to the cranium.

In Example 20, the subject matter of any one or more of Examples 17-19 optionally include wherein the inner implant and the outer implant are manufactured through selective laser sintering.

In Example 21, the subject matter of Example any one or more of Examples 17-20 includes, wherein the outer implant is U-shaped.

Example 22 is a hard tissue implant assembly comprising: an outer portion securable to a cranium of a patient; and an inner portion engageable with the outer portion to nest the inner portion within the outer portion, the inner portion securable to the outer portion.

In Example 23, the subject matter of Example 22 includes, wherein an inner edge of the outer implant is beveled and an outer edge of the inner implant is beveled complimentary to the beveled inner edge of the outer implant to allow the inner implant to nest in the outer implant.

In Example 24, the subject matter of Example 23 includes, a plurality of fasteners securable to the inner portion and the outer portion to secure the inner portion to the outer portion and to secure the outer portion to the cranium.

In Example 25, the subject matter of Example 24 includes, wherein the outer implant is trimmable to reduce an overall shape and size of the outer implant, the portion of the outer implant securable to the cranium, and the inner implant securable to the portion of the outer implant and to the cranium.

In Example 26, the subject matter of Examples 22-25 includes, D printing.

In Example 27, the subject matter of Example 26 includes, wherein the inner implant and the outer implant are manufactured through selective laser sintering.

In Example 28, the subject matter of Examples 22-27 includes, wherein the outer implant is U-shaped.

In Example 29, the subject matter of Examples 22-28 includes, wherein the inner implant is securable to a portion of the outer implant and to the cranium.

In Example 30, the subject matter of any one or more of Examples 1-29 optionally include a method for repairing any bone.

In Example 31, the apparatuses or method of any one or any combination of Examples 1-30 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples,” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of replacing a portion of a cranium with a hard tissue implant, the method comprising:
resecting a first portion of the cranium to define a first margin region;
analyzing the first portion of the cranium to determine if the first margin region is free of cancerous cells;
resecting a second portion of the cranium to define a second margin region that is larger than the first margin region when the first margin region is not free of cancerous cells;
securing an outer implant to the cranium at the second margin region; and
securing an inner implant to the outer implant.

2. The method of claim 1, wherein the outer implant is secured to the inner implant using a plurality of fasteners.

3. The method of claim 1, further comprising:
nesting the inner implant in the outer implant.

4. The method of claim 3, wherein an inner edge of the outer implant is beveled and an outer edge of the inner implant is beveled complimentary to the beveled inner edge of the outer implant to allow the inner implant to nest in the outer implant.

5. The method of claim 1, further comprising:
trimming the outer implant based on the analysis; and
securing the trimmed outer implant to the cranium.

6. The method of claim 5, further comprising:
securing the inner implant to the trimmed outer implant and to the cranium.

7. The method of claim 5, wherein the outer implant is U-shaped.

8. The method of claim 1, further comprising:
marking, using the outer implant as a guide, the cranium for resecting the first portion of the cranium to the first margin region.

9. The method of claim 1, wherein the inner implant and the outer implant are manufactured through selective laser sintering.

10. A method of replacing a portion of a cranium with a hard tissue implant, the method comprising:
marking the cranium for resecting a first portion of the cranium to define a first margin region using an inner edge of an outer implant as a guide;
resecting the first portion of the cranium to the first margin region;
analyzing the first portion of the cranium to determine if the first margin region is free of cancerous cells; and
securing an inner implant to the cranium when the first margin region is free of cancerous cells.

11. The method of claim 10, further comprising
resecting a second portion of the cranium to define a second margin region that is larger than the first margin region when the first margin region is not free of cancerous cells; and
securing an outer implant to the cranium at the second margin region.

12. The method of claim 11, further comprising:
securing an inner implant to the outer implant when the first margin region is not free of cancerous cells.

13. The method of claim 12, wherein the outer implant is secured to the inner implant using a plurality of fasteners.

14. The method of claim 12, further comprising:
nesting the inner implant in the outer implant.

15. The method of claim 14, wherein an inner edge of the outer implant is beveled and an outer edge of the inner implant is beveled complimentary to the beveled inner edge of the outer implant to allow the inner implant to nest in the outer implant.

16. The method of claim 15, wherein the outer implant is U-shaped.

17. A hard tissue implant assembly comprising:
an outer portion securable to a cranium of a patient, the outer portion including an inner edge that is beveled; and
an inner portion including a beveled outer edge engageable with the inner edge of the outer portion to nest the inner portion within the outer portion, the inner portion securable to the outer portion.

18. The implant assembly of claim 17, further comprising:
a plurality of fasteners securable to the inner portion and the outer portion to secure the inner portion to the outer portion and to secure the outer portion to the cranium.

19. The implant assembly of claim 17, wherein the outer implant is trimmable to reduce an overall shape and size of the outer implant, the portion of the outer implant securable to the cranium, and the inner implant securable to the portion of the outer implant and to the cranium.

20. The implant assembly of claim 17, wherein the inner implant and the outer implant are manufactured through selective laser sintering.
